SEQUENCE LISTING (5 sequences)

SEQ ID NO: 1 - DNA, 1475 bp, unidentified
  source 1..1475: mol_type = genomic DNA
tggcgcggcggtgtgctatacatgcagtcgtacgcacgggcccaactgattgatggtgcttgcacctgattgacgatggatcaccagtgagtggcggacgggtgagtaacacgtaggtaacctgccccggagcgggggataacatttggaaacagatgctaataccgcataacaacaaaagccacatggcttttgtttgaaagatggctttggctatcactctgggatggacctgcggtgcattagctagttggtaaggtaacggcttaccaaggcgatgatgcatagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttggagaagaacgtgcgtgagagtaactgttcacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgtttggagggtttccgcccttcagtgccggagctaacgcattaagcactccgcctgggagtacgaccgcaaggttgaaactcaaagaattgacgggggcccgcacaagcgtggagcatgtggttgatttcaagctacgcgaagaccttaccaggtcttgacatcttgcgctaaccttagaggataaggcgttcccttcggggacgcaatgacaggtggtgcatggtcgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcagatcatcatgccccttatgacctgggctacacacgtgctacaatggacggtacaacgagtcgcaagctcgcgagagtaagctaatctcttaaagccgttctcagttcggactgtaggctgcaactcgcctacacgaagtcggaatcgctagtaatcgcggatcagcatgccgcggtgaatacgttcccgggccttgtacacaccgcccgtcacaccatgggagtttgtaacgcccaaagtcggtggcctaacctttatggaggagccgctaagcgacagtgcg

SEQ ID NO: 2 - DNA, 1475 bp, unidentified; identical to SEQ ID NO: 1
  source 1..1475: mol_type = genomic DNA

SEQ ID NO: 3 - DNA, 1449 bp, unidentified
  source 1..1449: mol_type = genomic DNA
gggatgggcggcgtgctatacatgcagtcgagcgagcttgcctagatgaatttggtgcttgcaccaaatgaaactagatacaagcgagcggcggacgggtgagtaacacgtgggtaacctgcccaagagactgggataacacctggaaacagatgctaataccggataacaacactagacgcatgtctagagtttaaaagatggttctgctatcactcttggatggacctgcggtgcattagctagttggtaaggtaacggcttaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtgcgaccgcaaggttgaaagtcaaaggatttgacgggggcccgcgcgagcggtggagcgtgtggtttagttcgaagcaacgcgaggagcctgtaccagtcttgacgtccagtgcaaaccgaggagattaggagtacctgtcgtcagctcgtgtcgtgagatgttgggttaagtcccgcaacgagcgcaacccttgtcattagttgccatcattaagttgggcactctaatgagactgccggtgacaaaccggaggaaggtggggatgacgtcaagtcatcatgccccttatgacctgggctacacacgtgctacaatggacggtacaacgagaagcgaacctgcgaaggcaagcggatctctgaaagccgttctcagttcggactgtaggctgcaactcgcctacacgaagctggaatcgctagtaatcgcggatcagcacgccgcggtgaatacgttcccgggccttgtacacaccgcccgtcacaccatgagagtctgtaacacccaaagccggtgggataacctttataggagtcagccgtctaagtagacagggtgggt

SEQ ID NO: 4 - DNA, 1816 bp, unidentified
  source 1..1816: mol_type = genomic DNA
ggcatggcggcgtgctatacatgcagtcgagcgagcgtgaaccaacagattcacttcggtgatgacgttgggaacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttggggaggtttccgcctctcagtgctgcagctaacgcattaagcactccgcctggggagtacggaccgcaaggttgagactcaaaggaattgacgggggcccgcacaagcggtggagcgatgtggtttagtgtcgaagcaacgcgaagagccttaccaggtcttgacgtctagtgcaatccgggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgcaatccgtagagatacggagttcccttcggggacactaagacaggtggtgcatggctgtcgtcagctcgtgtcgtgagatgttgggttaagtcccgcaacgagcgcaacccttgtcattagttgccagcattaagttgggcactctaatgagactgccggtgacaaaccggaggaaggtggggatgacgtcaagtcatcatgccccttatgacctgggctacacacgtgctacaatggacagtacaacgaggagcaagcctgcgaaggcaagcgaatctcttaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaacctcggaaggagccgtctaagcagcgttgcg

SEQ ID NO: 5 - DNA, 1439 bp, unidentified
  source 1..1439: mol_type = genomic DNA
gaggcatgtggcgtgcttaccatgcagtcgacgggatccctggcagcttgctgtcggggtgagagtggcgaacgggtgagtaatgcgtgaccaacctgccctgtgcaccggaatagctcctggaaacgggtggtaataccggatgctccgctccatcgcatggtggggtgggaaatgcttttgcggcatgggatggggtcgcgtcctatcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaatacgtagggtgcgagcgttatccggatttattgggcgtaaagggctcgtaggcggttcgtcgcgtccggtgtgaaagtccatcgcctaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggccctttccacgggtcccgtgtcggagccaacgcgttaagcatcccgcctgggagtacggccgcaaggctaaaactcaaagaaattgacggggcccgcacaagcggcggagcatgcggattaattcgatgcaacgcgaagaaccttacctgggcttgacatgtgccggatcgccgtggagacacggtttcccttcggggccggttcacaggtggtgcatggtcgtcgtcagctcgtgtcgtgagatgttgggttaagtcccgcaacgagcgcaaccctcgccgcatgttgccagcgggtgatgccgggaactcatgtgggaccgccggggtcaactcggaggaaggtggggatgacgtcagatcatcatgccccttacgtccagggcttcacgcatgctacaatggccggtacaacgcggtgcgacacggtgacgtggggcggatcgctgaaaaccggtctcagttcggatcgcagtctgcaactcgactgcgtgaaggcggagtcgctagtaatcgcggatcagcaacgccgcggtgaatgcgttcccgggccttgtacacaccgcccgtcaagtcatgaaagtgggtagcacccgaagccggtggcccgacccttgtggggggagccgtctaagtagactcatg

(19) United States
(12) Patent Application Publication
(10) Pub. No.: US 2025/0249050 A1
(43) Pub. Date: Aug. 7, 2025

(54) USE OF A FORMULATION COMPRISING PROBILTICS AND METABOLITES THEREOF (POSTBIOTICS) IN THE PREPARATION OF A PRODUCT FOR ALLEVIATING COLORECTITIS

(71) Applicant: INSTITUTE OF MICROBIOLOGY, GUANGDONG ACADEMY OF SCIENCES (GUANGDONG DETECTION CENTER OF MICROBIOLOGY, Guangdong (CN)

(72) Inventor: Liwei XIE, Guangdong (CN)

(73) Assignee: INSTITUTE OF MICROBIOLOGY, GUANGDONG ACADEMY OF SCIENCES (GUANGDONG DETECTION CENTER OF MICROBIOLOGY, Guangdong (CN)

(21) Appl. No.: 18/856,886

(22) PCT Filed: Jul. 4, 2022
(86) PCT No.: PCT/CN2022/103560

§ 371 Date: Nov. 26, 2024

(30) Foreign Application Priority Data
Apr. 15, 2022 (CN) 202210397574.1

(51) Int. Cl. A61K 35/747; A61K 35/00; A61K 35/745; A61P 1/00; C12Q 1/06

(52) U.S. Cl. CPC A61K 35/747; A61K 35/745; A61P 1/00; C12Q 1/06; A61K 2035/115

(57) ABSTRACT

The invention relates to core components from five strains of independently isolated, identified and patent-deposited probiotics and fermentation metabolites thereof (postbiotics), as well as their use as a protector in alleviating dextran sulfate sodium (DSS) induced-colitis in mice. Further disclosed the mechanism of the probiotics and fermentation metabolites thereof (postbiotics) to alleviate colitis in mice.

### TECHNICAL FIELD

The present invention generally relates to microbial technology field, particularly to use of a formulation comprising probiotics and metabolites thereof (postbiotics) in the preparation of a product for alleviating colorectitis.

### RELATED ART

Inflammatory bowel disease (IBD) has become a global public health issue due to its high incidence rate and low cure rate. Although the pathogenesis of this disease is unclear, the potential association between gut microbiota and inflammatory signals has been established, which provides a breakthrough for the study of this disease. Probiotics, especially Lactobacillus and Bifdobacterium, have recently become a hot spot in the treatment of IBD due to their safety, effectiveness, and strong ability to regulate the microbiota. However, it is rarely reported about the evaluation of the effects of multi strain combinations and metabolites thereof on IBD in most studies. In this study, we studied the protection of laboratory isolated and patented probiotics, including Lactobacillus reuteri, Lactobacillus gasseri, Lactobacillus acidophilus, and Bifidobacterium lactis, and metabolites thereof, on mice with dextran sulfate sodium (DSS)—induced colitis. The results demonstrated that the mix strains and metabolites thereof not only improved the disease phenotype, such as reducing weight changes, DAI scores, and histopathological scores, but also improved the composition of gut microbiota, as defined by increase of probiotics and reduction of pathogens, and the effects were better than that of a single strain. In addition, a complex and tight interaction network between gut microbiota and phenotype was discovered. In summary, the probiotic strains and metabolites thereof function as a protector on DSS-induced colitis models by mitigating inflammation and regulating disordered microbiota, and the intervention effect of mix strains and metabolites thereof is better than that of a single strain, providing a strategy for the clinical treatment of colitis with probiotics in the future.

### SUMMARY OF INVENTION

The present invention provides five independently isolated and identified probiotics, fermented metabolites thereof, and microbial composition formulations for clinical prevention and treatment of colorectal-associated inflammatory enteritis.

The five independently isolated probiotics in the present invention is listed below:

Lactobacillus reuteri PLBK®1 (GDMCC No: 60828) was deposited at the Guangdong Microbial Culture Collection Center (GDMCC) on Oct. 24, 2019, location: 5/F, Building 59, No. 100 Xianlie Central road, Yuexiu District, Guangzhou, China, 510070.

Lactobacillus reuteri PLBK®2 (GDMCC No: 60829) was deposited at the Guangdong Microbial Culture Collection Center (GDMCC) on Oct. 24, 2019, location: 5/F, Building 59, No. 100 Xianlie Central road, Yuexiu District, Guangzhou, China, 510070.

Lactobacillus gasseri PLBK®3 (GDMCC No: 60830) was deposited at the Guangdong Microbial Culture Collection Center (GDMCC) on Oct. 24, 2019, location: 5/F, Building 59, No. 100 Xianlie Central road, Yuexiu District, Guangzhou, China, 510070.

Lactobacillus acidophilus PLBK®4 (GDMCC No: 60831) was deposited at the Guangdong Microbial Culture Collection Center (GDMCC) on Oct. 24, 2019, location: 5/F. Building 59, No. 100 Xianlie Central road, Yuexiu District, Guangzhou, China, 510070.

Bifdobacterium lactis PLBK®5 (GDMCC No: 60832) was deposited at the Guangdong Microbial Culture Collection Center (GDMCC) on Oct. 24, 2019, location: 5/F, Building 59, No. 100 Xianlie Central road, Yuexiu District, Guangzhou, China, 510070.

The present invention provides use of one or more of Lactobacillus reuteri PLBK®1, Lactobacillus reuteri PLBK®2, Lactobacillus gasseri PLBK®3, Lactobacillus acidophilus PLBK®4 and Bifdobacterium lactis PLBK®5, fermentation cultures or metabolites thereof in the manufacturing of a product for alleviating colorectitis.

The product for alleviating colorectitis can be food, healthcare products, or drugs.

Preferably, the present invention provides use of the mixture of five strains, Lactobacillus reuteri PLBK®1. Lactobacillus reuteri PLBK®2, Lactobacillus gasseri PLBK®3, Lactobacillus acidophilus PLBK®4 and Bifdobacterium lactis PLBK®5, the mixture of fermentation cultures or the mixture of metabolites thereof in the preparation of a product for alleviating colorectitis.

Preferably, Lactobacillus reuteri PLBK®1, Lactobacillus PLBK®2. Lactobacillus gasseri PLBK®3, Lactobacillus acidophilus PLBK®4 and Bifdobacterium lactis PLBK®5 are mixed at a quantity ratio of 1:1:1:1:1.

More preferably, the mixture of Lactobacillus reuteri PLBK®1, Lactobacillus reuteri PLBK®2, Lactobacillus gasseri PLBK®3, Lactobacillus acidophilus PLBK®4 and Bifdobacterium lactis PLBK®5 is a bacterial solution with the content of bacteria is 10^{\hat{ }9} cfu/ml and above. More preferably, the content of bacteria is 10^{\hat{ }9} cfu/ml.

The second object of the present invention is to provide use of one or more of Lactobacillus reuteri PLBK®1, Lactobacillus reuteri PLBK®2, Lactobacillus gasseri PLBK®3, Lactobacillus acidophilus PLBK®4 and Bifdobacterium lactis PLBK®5, fermentation cultures or metabolites thereof in the preparation of a product for increasing the relative abundance of Bacteroidaceae Bacteroides and Lachnospiraceae Ruminococcus in the intestine.

The third object of the present invention is to provide use of Bacteroidaceae Bacteroides and/or Lachnospiraceae Ruminococcus as biomarkers for identifying the recovery of intestinal inflammation.

The fourth object of the present invention is to provide use of a preparation for detecting the content of Bacteroidaceae Bacteroides and/or Lachnospiraceae Ruminococcus in the preparation of a preparation for identifying the recovery of intestinal inflammation.

The present invention relates to core components from five strains of independently isolated, identified and patent-deposited probiotics and fermentation metabolites thereof (postbiotics), as well as their use as a protector in alleviating dextran sulfate sodium (DSS) induced-colitis in mice. Also disclosed the mechanism of the probiotics and fermentation metabolites thereof (postbiotics) to alleviate colitis in mice. The use of the present invention can achieve the effects of reducing macrophage infiltration, reducing intestinal inflammation, regulating gut microbiota microenvironment and accelerating intestinal mucosa repair, which is promising to be applicable in the clinical prevention and treatment of colorectal-associated inflammatory enteritis.

Lactobacillus reuteri PLBK®1 (GDMCC No: 60828) was deposited at the Guangdong Microbial Culture Collection Center (GDMCC) on Oct. 24, 2019, location: 5/F, Building 59, No. 100 Xianlie Central road, Yuexiu District, Guangzhou, China, 510070.

Lactobacillus reuteri PLBK®2 (GDMCC No: 60829) was deposited at the Guangdong Microbial Culture Collection Center (GDMCC) on Oct. 24, 2019, location: 5/F, Building 59, No. 100 Xianlie Central road, Yuexiu District, Guangzhou, China, 510070.

Lactobacillus gasseri PLBK®3 (GDMCC No: 60830) was deposited at the Guangdong Microbial Culture Collection Center (GDMCC) on Oct. 24, 2019, location: 5/F, Building 59, No. 100 Xianlie Central road, Yuexiu District, Guangzhou, China, 510070.

Lactobacillus acidophilus PLBK®4 (GDMCC No: 60831) was deposited at the Guangdong Microbial Culture Collection Center (GDMCC) on Oct. 24, 2019, location: 5/F, Building 59, No. 100 Xianlie Central road, Yuexiu District, Guangzhou, China, 510070.

Bifdobacterium lactis PLBK®5 (GDMCC No: 60832) was deposited at the Guangdong Microbial Culture Collection Center (GDMCC) on Oct. 24, 2019, location: 5/F, Building 59, No. 100 Xianlie Central road, Yuexiu District, Guangzhou, China, 510070.

### BRIEF DESCRIPTION OF DRAWINGS

FIG. 1 shows a strain gene circular functional map of the complete genome sequencing of Lactobacillus reuteri PLBK®1;

FIG. 2 shows a strain gene circular functional map of the complete genome sequencing of Lactobacillus reuteri PLBK®2;

FIG. 3 shows a strain gene circular functional map of the complete genome sequencing of Lactobacillus gasseri PLBK®3;

FIG. 4 shows a strain gene circular functional map of the complete genome sequencing of Lactobacillus acidophilus PLBK®4;

FIG. 5 shows a strain gene circular functional map of the complete genome sequencing of Bifdobacterium lactis PLBK®5;

FIG. 6 shows the evolutionary map of (A) Lactobacillus reuteri PLBK®1 and Lactobacillus reuteri PLBK®2, (B) Lactobacillus gasseri PLBK®3, (C) Lactobacillus acidophilus PLBK®4, and (D) Bifdobacterium lactis PLBK®5;

FIG. 7 shows the identification of fermentation metabolites of the five probiotic strains;

FIG. 8 shows the identification of purified fermentation metabolites of the five probiotic strains;

FIG. 9 shows that the mix strains mitigates the pathological symptoms of DSS-induced experimental colitis.

FIG. 10 shows that the strains and metabolites thereof could restore inflammatory damage to the colon mucosa.

FIG. 11 shows that mix probiotic strains can regulate the gut microbiota composition of DSS-induced colitis model.

FIG. 12 shows that there is a strong correlation between gut microbiota and disease phenotype in colitis mice model.

FIG. 13 shows the identification of key microbial markers that interact with mix strains and metabolites thereof during the recovery of intestinal inflammation.

### DESCRIPTION OF EMBODIMENTS

It is intended that the following embodiments shall be interpreted as illustrative and not in a limiting sense. The protection scope of the present invention shall not be limited to the detailed embodiments below.

### Embodiment 1

### I. Experimental Method

### 1. Types of Probiotic Strains and Patent Deposit ID

The present invention obtained five probiotics through screen and isolation, and obtained Lactobacillus reuteri PLBK®1, Lactobacillus reuteri PLBK®2, Lactobacillus gasseri PLBK®3. Lactobacillus acidophilus PLBK®4, Bifdobacterium lactis PLBK®5 through 16S sequencing (16S full-length amplification primers, 16S_Forw: AGAGTTGATCGCTCG, 16S_Rev: GGTTACCTTGTTACGACTT), gene circular functional map of the complete genome sequencing (extraction of nucleic acid from strain fermentation sludge, set up of a library for next-generation sequencing, with Ilumina Hiseq X10 as the sequencing platform), and construction of evolutionary trees. The information in detail is listed below:

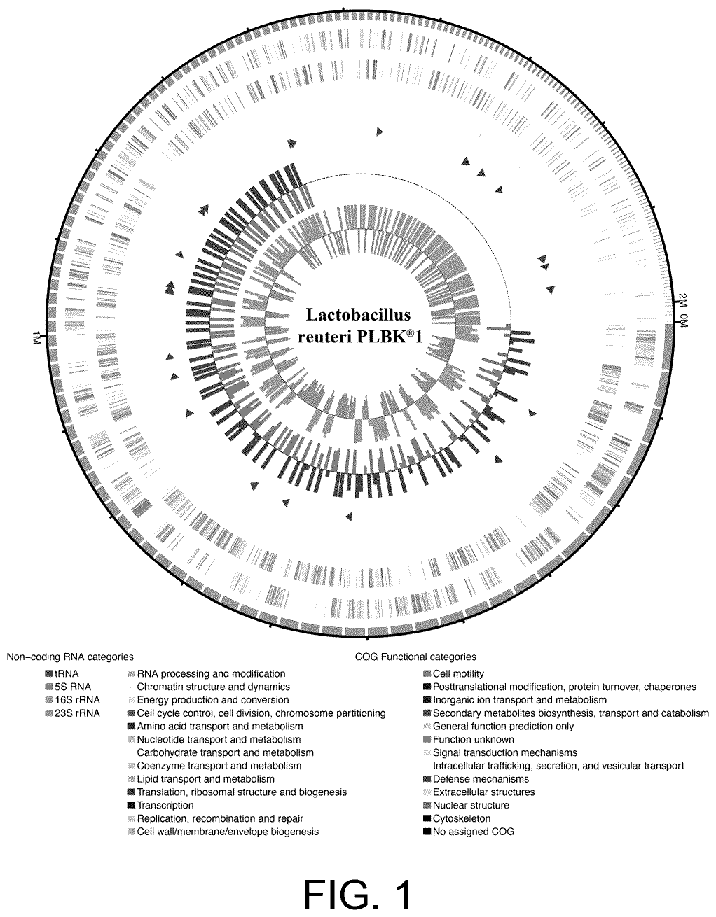

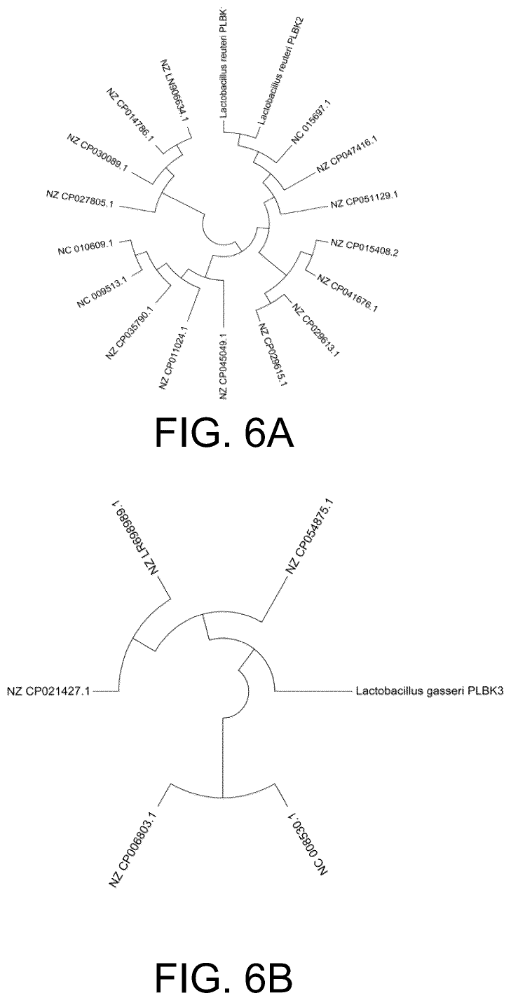

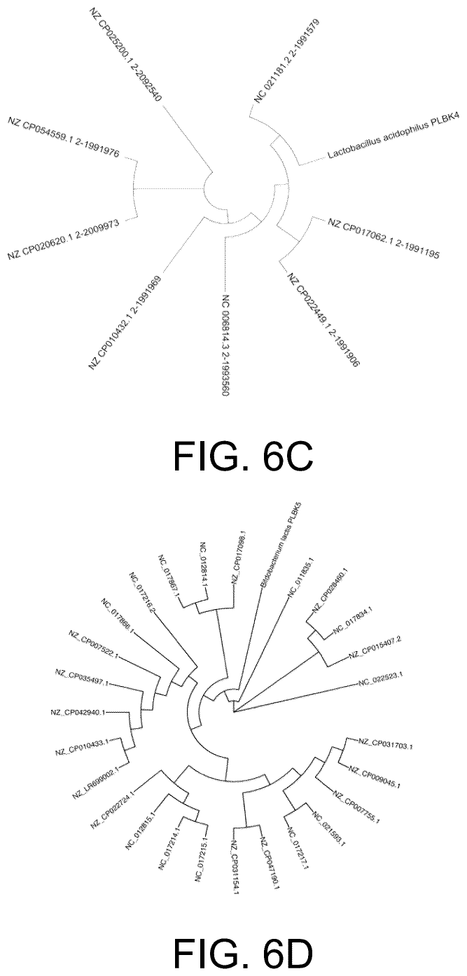

Lactobacillus reuteri PLBK®1 (GDMCC No: 60828) was deposited at the Guangdong Microbial Culture Collection Center (GDMCC) on Oct. 24, 2019, location: 5/F, Building 59, No. 100 Xianlie Central road, Yuexiu District, Guangzhou, China, 510070. The nucleotide sequence of its 16s rDNA is shown in SEQ ID NO.1, and the strain gene circular functional map of the complete genome sequencing is shown in FIG. 1, and the evolutionary map is shown in FIG. 6.

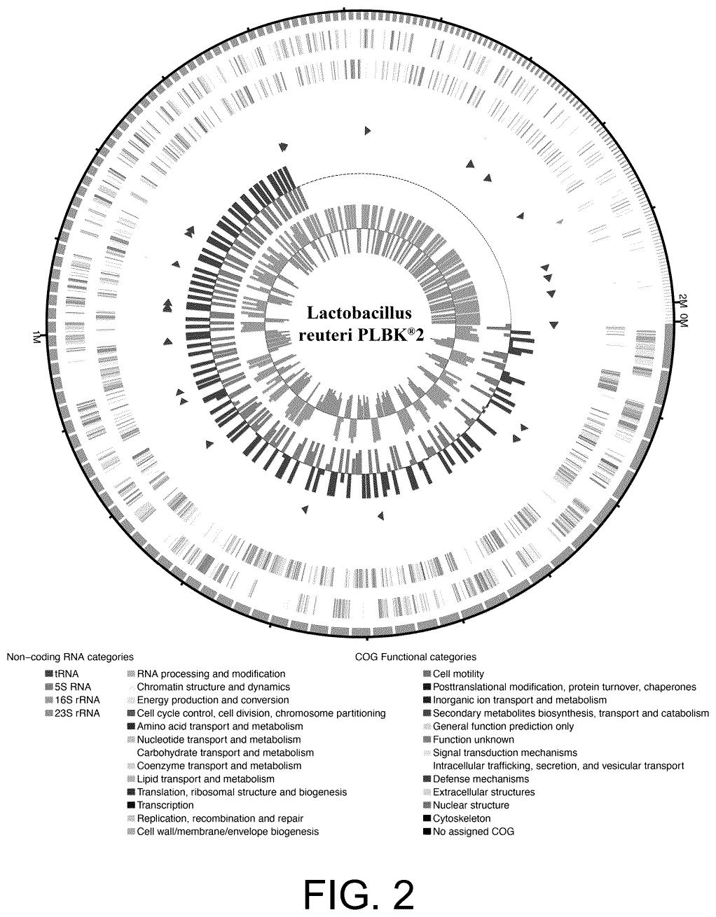

Lactobacillus reuteri PLBK®2 (GDMCC No: 60829) was deposited at the Guangdong Microbial Culture Collection Center (GDMCC) on Oct. 24, 2019, location: 5/F, Building 59, No. 100 Xianlie Central road, Yuexiu District, Guangzhou, China, 510070. The nucleotide sequence of its 16s rDNA is shown in SEQ ID NO.2, and the strain gene circular functional map of the complete genome sequencing is shown in FIG. 2, and the evolutionary map is shown in FIG. 6.

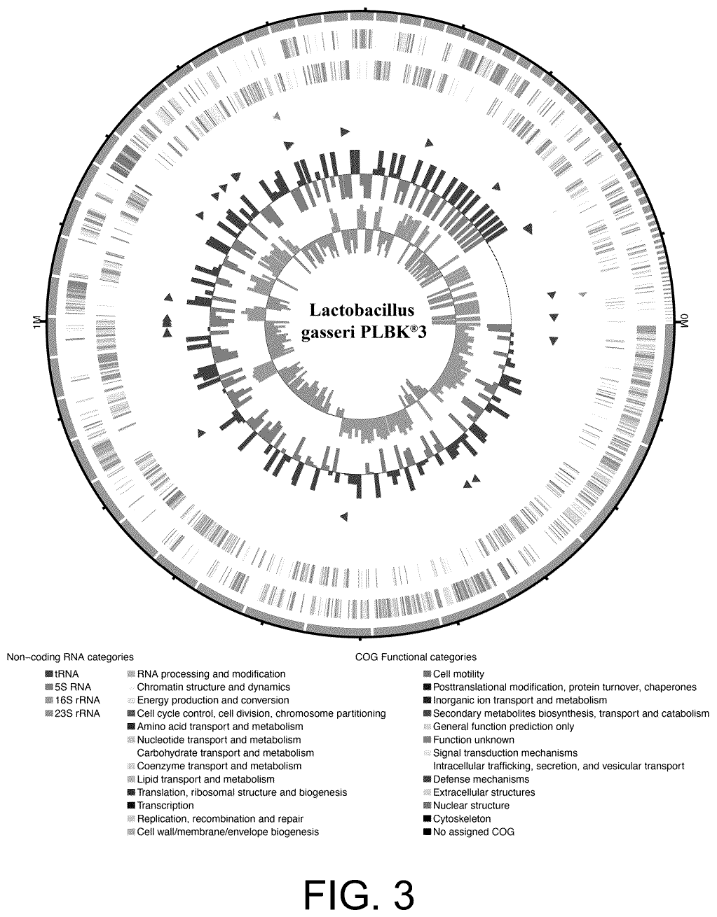

Lactobacillus gasseri PLBK®3 (GDMCC No: 60830) was deposited at the Guangdong Microbial Culture Collection Center (GDMCC) on Oct. 24, 2019, location: (5/F, Building 59, No. 100 Xianlie Central road, Yuexiu District, Guangzhou, China, 510070. The nucleotide sequence of its 16s rDNA is shown in SEQ ID NO.3, and the strain gene circular functional map of the complete genome sequencing is shown in FIG. 3, and the evolutionary map is shown in FIG. 6.

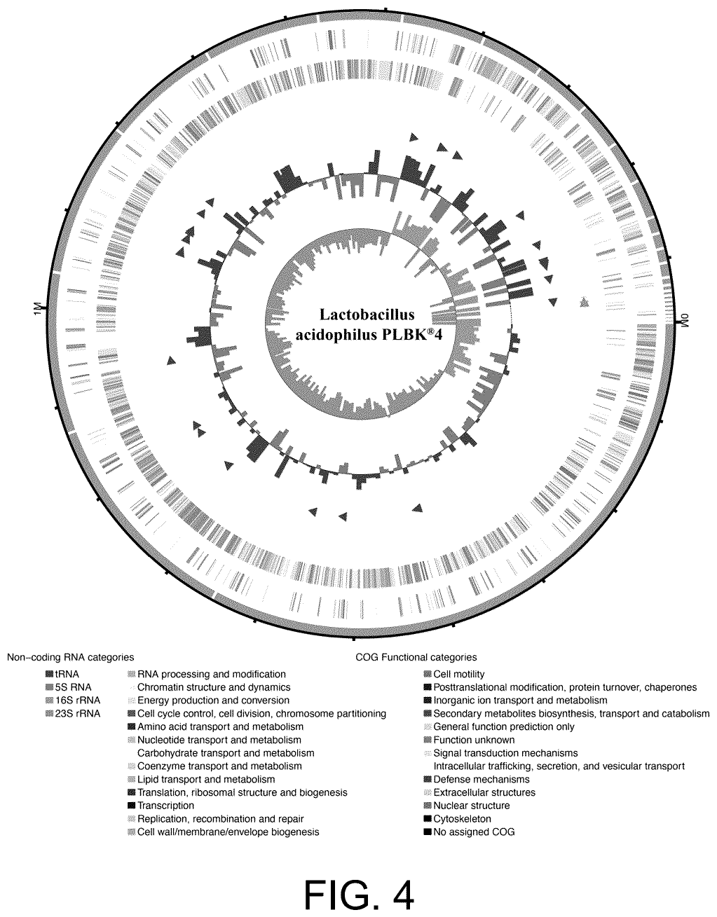

Lactobacillus acidophilus PLBK®4 (GDMCC No: 60831) was deposited at the Guangdong Microbial Culture Collection Center (GDMCC) on Oct. 24, 2019, location: 5/F, Building 59, No. 100 Xianlie Central road, Yuexiu District, Guangzhou, China, 510070. The nucleotide sequence of its 16s rDNA is shown in SEQ ID NO.4, and the strain gene circular functional map of the complete genome sequencing is shown in FIG. 4, and the evolutionary map is shown in FIG. 6.

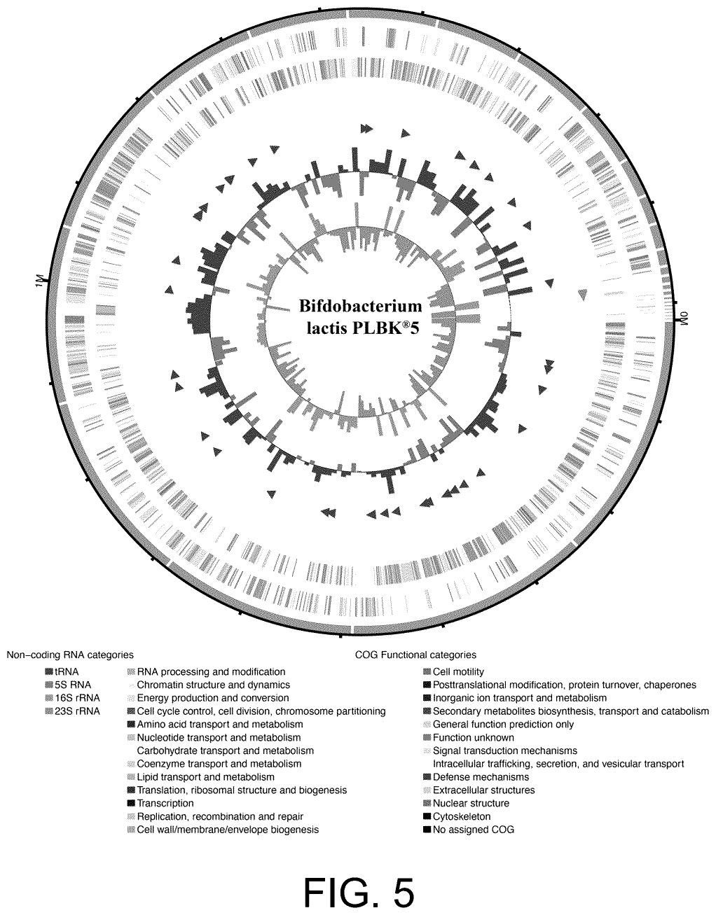

Bifdobacterium lactis PLBK®5 (GDMCC No: 60832) was deposited at the Guangdong Microbial Culture Collection Center (GDMCC) on Oct. 24, 2019, location: 5/F, Building 59, No. 100 Xianlie Central road, Yuexiu District, Guangzhou, China, 510070. The nucleotide sequence of its 16s rDNA is shown in SEQ ID NO.5, and the strain gene circular functional map of the complete genome sequencing is shown in FIG. 5, and the evolutionary map is shown in FIG. 6.

### 2. Fermentation Medium

Optimized MRS medium for Lactobacillus rhamnosus, Lactobacillus gasseri, and Lactobacillus acidophilus: peptone (10 g/L), beef extract powder (8 g/L), yeast extract powder (4 g/L), glucose (20 g/L), dipotassium hydrogen phosphate (2 g/L), diammonium hydrogen citrate (2 g/L), sodium acetate (5 g/L), magnesium sulfate (0.2 g/L), manganese sulfate (0.04 g/L), and water as the solvent. The specific preparation method is to mix the components evenly, and sterilize it for later use.

Optimized Bifidobacterium medium for Bifdobacterium lactis: peptone (15 g/L), glucose (20 g/L), yeast extract powder (2 g/L), soluble starch (0.5 g/L), sodium chloride (5 g/L), L-cysteine (0.5 g/L), tomato extract powder (5 g/L), liver extract powder (2 g/L),and water as the solvent. The specific preparation method is to mix the components evenly, and sterilize it for later use.

3. Probiotic fermentation and preparation process: Lactobacillus or Bifidobacterium was inoculated on 5 ml sterilized MRS liquid medium or Bifidobacterium liquid medium, respectively, anaerobically grew for 24 hours, then transferred 5ml of bacterial solution to 1L of medium for anaerobic fermentation and amplification for another 48 hours. After fermentation, the bacterial sludge was harvested by centrifugation, and the filtered supernatant was obtained by filtering the supernatant of the bacterial solution through a 0.22 μm filter membrane. After obtaining the cfu of probiotics by plate counting method, resuspended the bacterial slurry with filtered supernatant to obtain 1029 cfu/ml of single strain bacterial solution. Then, mixed the bacterial solutions from the five single strains by volume ratio PLBK®1: PLBK®2: PLBK®3: PLBK®4: PLBK®5=1:1:1:1:1 to obtain a mix probiotic bacterial solution for the following experiments.

4. Preparation process of probiotic metabolites: the fermentation metabolites of each strain were obtained by centrifugation of the strain fermentation broth and filtration of the supernatant (0.22 μm). Then, the fermentation metabolites of five strains were mixed in equal proportions by volume to obtain the mix probiotic fermentation metabolites, which were stored at 4° C.

### 5. Purification Process of Probiotic Metabolites

A. Poured 500 ml of the supernatant of the probiotic fermentation solution, which was

filtered through a 0.22 μm filter membrane, into a separating funnel, and then poured 500 ml of ethyl acetate into it;

B. Shook the separating funnel horizontally for 2 minutes to thoroughly mix the two liquids;

C. Moved the separating funnel to a fume hood and kept it rested at room temperature for 5 minutes so that the liquid was allowed to fully separate into layers;

D. Repeated steps B and C three to four times, and at that moment, the upper liquid was observed to be yellow brown transparent and the lower liquid was brown. (The upper liquid was in the oil phase: substances soluble in ethyl acetate from the supernatant; the lower liquid was in the water phase: substances insoluble in ethyl acetate from the supernatant).

E. Placed a 500 ml triangular flask at the bottom of the separating funnel to receive the lower liquid;

F. Opened the upper piston of the separating funnel to maintain consistent pressure inside and outside the funnel, then unscrew the lower piston of the separating funnel to discharge the lower liquid into the triangular flask. At that moment, a few amount of upper liquid can also be discharged into the triangular flask to remove interference from liquid interface.

G. After the discharge is completed, closed the lower piston. Poured the lower liquid from the upper opening into a rotary flask;

H. Connected the rotary flask to the rotary evaporator, soaked ¼ of the rotary flask in a water bath, adjusted the temperature of the water bath to 45° C., and turned on the main switch of the rotary evaporator.

I. When the liquid inside the rotary flask evaporated until no more decrease, turned off the main switch and removed the rotary flask; Added 2-3 ml of ethyl acetate to the rotary flask, mixed thoroughly, and transferred the liquid from the rotary flask to the sample bottle with a pipette:

J. Connected the sample bottle to the rotary evaporator, soaked ¼ of the sample bottle in a water bath, adjusted the temperature of the water bath to 45° C., and turned on the main switch of the rotary evaporator.

K. When the liquid in the sample bottle evaporated until no more decrease, turned off the main switch and removed the sample bottle. The liquid in the bottle was the crude substance extracted from 500 ml of the supernatant of the bacterial solution.

The concentrated metabolites (aqueous phase) of each bacterium were obtained thereby.

6. Construction of colitis mice model: a colitis model was created in 14-week-old C57BL/6J mice by adding 2.5% dextran sulfate sodium (DSS, LOT NO: S2839, molecular weight of 36000-50000 Da; from MP Biomedicals LLC. Solon, Ohio, USA) into their sterile drinking water and allowing them freely drinking for 7 days. Six experimental groups (Lactobacillus rhamnosus PLBK®1—designated as LR1, Lactobacillus reuteri PLBK®2—designated as LR2, Lactobacillus delbrueckii PLBK®3—LG, Lactobacillus acidophilus PLBK®4—designated as LA, Bifidobacterium lactis PLBK®5—designated as BL, and mix probiotic group MIX) and a control group (CONTROL) were established. That is, the treatment groups were for five single strains bacterial solution and one mix probiotic bacterial solution, and the control group was a PBS intragastic group, with 6-10 mice in each group. After DSS modeling, 200 μL of bacterial solution was intragastically administered every day, once a day, for a total of 14 days. The weight changes and status of the mice were recorded daily, and feces were collected on Day 8 and Day 14. All mice were sacrificed on Day 15, and the colon tissue was taken out for photography and length measurement. Additional intestinal tissue was cut for formalin fixation. In the bacterial solution intervention experiment, the remaining intestinal contents were scraped off with glass slides for subsequent experimental operations.

7. H&E staining: mice colon was subjected to fixation, dehydration, embedding, sectioning, and melting at 68° C. for 50 minutes to dewaxing. The step for dewaxing and hydration is as follows: dewaxing of toluene dewaxing I for 5 minutes; dewaxing of Xylene dewaxing II for 5 minutes; dewaxing of Xylene dewaxing III for 5 minutes; dewaxing of 100% ethanol for 2 minutes; hydration of 95% ethanol I for 2 minutes; hydration of 95% ethanol II for 2 minutes; hydration of 80% ethanol for 2 minutes; Water rinse for 3 minutes, and then rinse in another box three times for 1 minute each. Then staining was performed with the following steps: immersion in hematoxylin for 2 minutes; rinse in tap water for 1 minute, 3-5 times (rinsed quickly sequentially in two staining boxes filled with tap water, and rinsed in running tap water in one dye box for 1 minute, repeated 5 times in another box); differentiation with 0.3% hydrochloric acid alcohol for several seconds (1-2s for intestine, 3 times for liver and BAT, and 1 time for iWAT, with 1 second for each time); rinse quickly with tap water; rinse twice for 1 min each in another box; bluing in bluing buffer for 10 minutes; eosin with 0.5% eosin aqueous solution: 4 hours for intestinal tract; 1-2 hours for liver: 5 hours for BAT, iWAT; dehydration of 80% ethanol I for 2 minutes; dehydration of 90% ethanol II for 2 minutes; dehydration of 95% ethanol II for 2 minutes;

dehydration of anhydrous ethanol I for 2 minutes; dehydration of anhydrous ethanol II for 2 minutes; clearing of Xylene I for 2 minutes; clearing of Xylene II for 2 minutes; coverslipping with neutral gum.

8. Histological scoring: the H&E-stained colon tissue was observed under a microscope and subjected to pathological scoring by observers who were blinded to the experimental details. The scoring criteria were a combined scoring of epithelium loss, crypt damage, depletion of goblet cells, and infiltration of inflammatory cells. The detail information are shown in Table 1 below:

TABLE 1
Histological scoring of dextran sulfate
sodium (DSS) induced-colitis

| Histological scoring | Score | Description |
|---|---|---|
| Epithelium loss | 0 | None |
| | 1 | 0.0-5.0% |
| | 2 | 5.0-10.0% |
| | 3 | Over 10.0% |
| Crypt damage | 0 | None |
| | 1 | 0.0-10.0% |
| | 2 | 10.0%-20.0% |
| | 3 | Over 20.0% |
| Depletion of goblet cells | 0 | None |
| | 1 | Mild |
| | 2 | Moderate |
| | 3 | Severe |
| Infiltration of inflammatory cells | 0 | None |
| | 1 | Mild |
| | 2 | Moderate |
| | 3 | Severe |

9. Fecal DNA extraction, sequencing library construction, and 16S rRNA sequencing: DNA was extracted from mice feces using MoBio PowerSoil DNA Extraction Kit (QIAGEN, USA), and its concentration was measured using a nanotitrator (ThermoFisher, USA). 50 ng of DNA was used to construct a 16S rRNA sequencing library, using Q5 High Fidelity DNA Polymerase (NEB) targeting the V3-V4 region of bacterial 16S rRNA (Forword primer: 5′-CCTACGGGNGGCWGCAG-3′; Reverse primer: 5′-GACTACHVGGGTATCTAATCC-3′), followed by purification with AMPure XP (Beck).

10. 16S rRNA amplicon sequencing and bioinformatics statistics: Qiime 2 standardized process was used for analysis, and high-quality amplicon sequence variants (ASVs) were obtained through DADA2 algorithm. According to the Silva database (published in December 2019), classification analysis was conducted and converted into relative abundance at the system, class, order, family, genus, and species levels. Microbial data with relative abundance below 0.001 or occurrence rate below 70% in all groups were filtered out to obtain core bacterial groups for further analysis. Alpha diversity, Beta diversity, co-occurrence network graph analysis, structural graph, random forest model, redundancy analysis (RDA), and PICRUSt analysis were conducted on the R software package EasyMicroPlot.

11. Statistical analysis: The statistical details and sample size of all experiments are described in the legend of each graph, and the results are presented as mean±standard error. The significance comparison between the two groups was conducted using a two tailed non paired student t-test. The significance test between multiple groups was conducted using one-way ANOVA followed by post hoc testing of Tukey's and LSD. The statistical significance is described in the legend as: *p<0.05. **p<0.01. ***p<0.001.

### II. Results

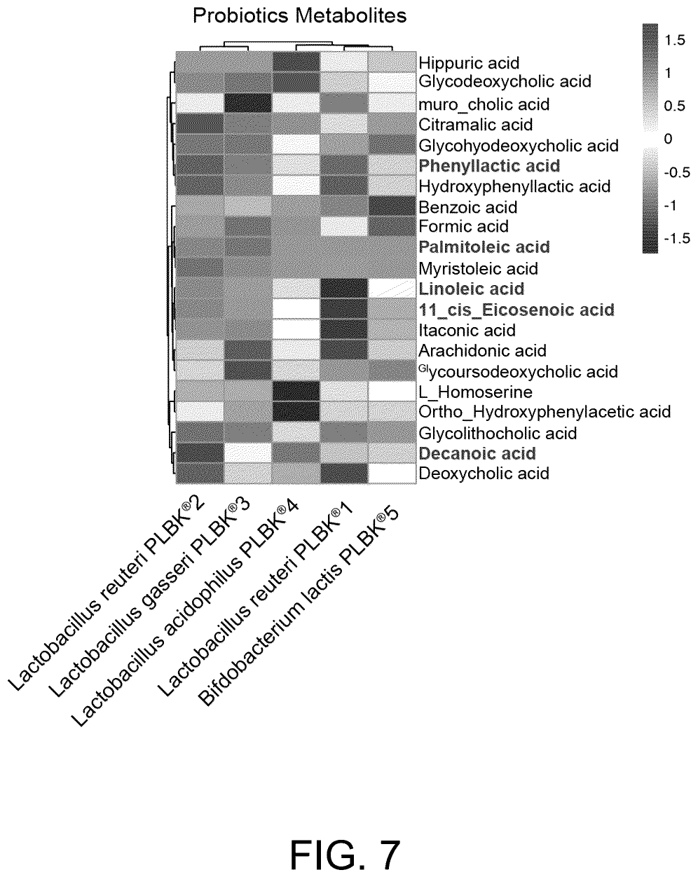

1. The identification results of fermentation metabolites of the five probiotic strains are shown in FIG. 7. After fermentation, the five strains of probiotics were filtered through a 0.22 μM filter membrane to obtain the fermentation supernatant. LC-MS/MS mass spectrometry was performed to analyse the metabolites in the supernatant, with MRS medium used as a blank control. The metabolites produced by probiotic fermentation were obtained by comparing metabolite composition and content with MRS blank control.

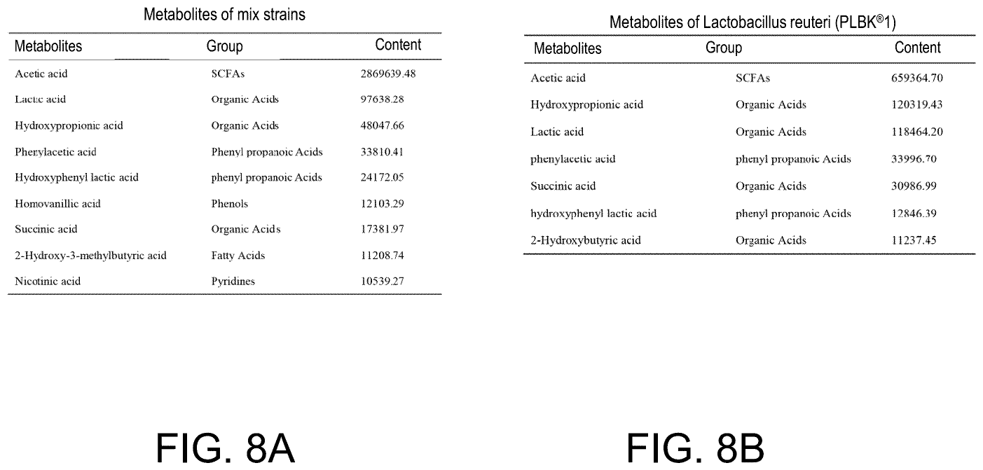

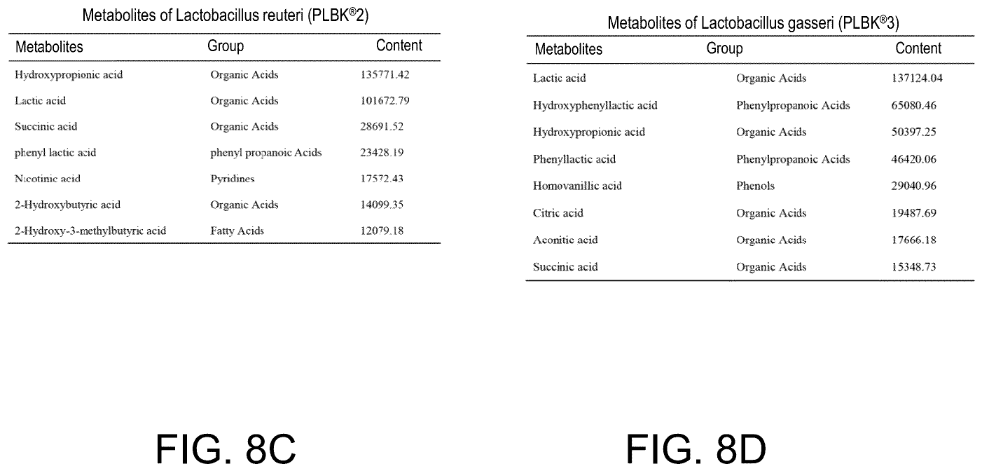

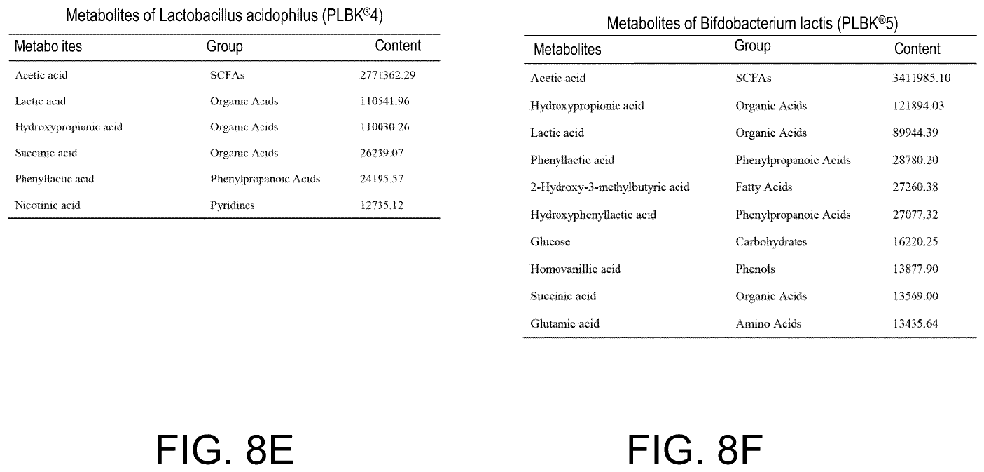

2. The identification of fermentation metabolites from the five purified probiotic strains is shown in FIG. 8. After fermentation, 5 strains of probiotics were filtered through 0.22 μM filtration and concentrated in ethyl acetate, and the metabolites in the lower layer (aqueous phase) were collected. The metabolites were concentrated at low temperature and evaporated to obtain 1000× concentration. LC-MS/MS mass spectrometry was performed to analyse the metabolites therein, and metabolites produced by probiotic fermentation were obtained by comparing metabolite composition and content with MRS blank control.

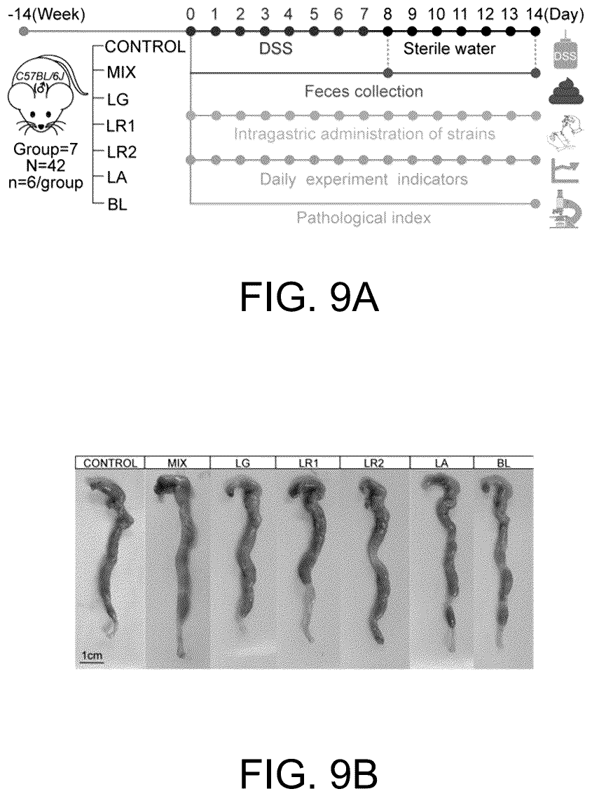

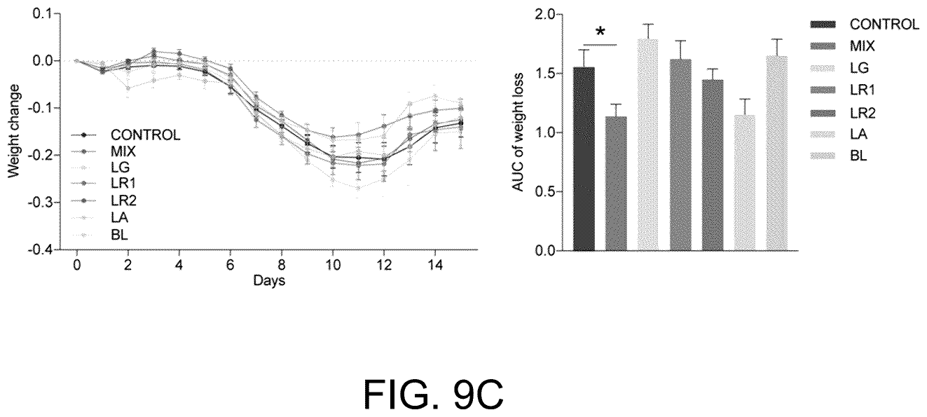

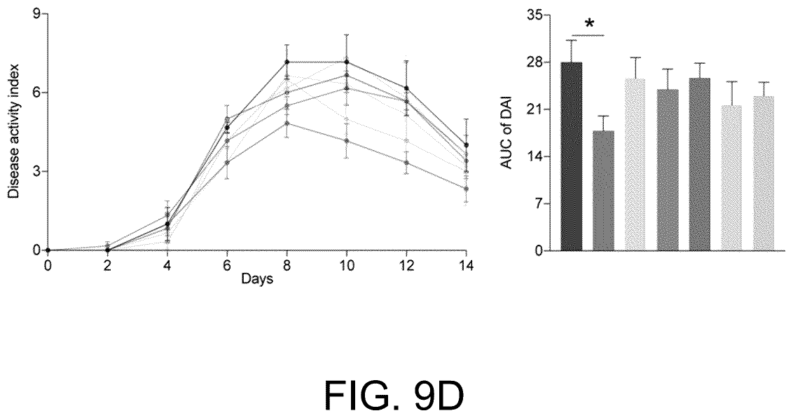

3. To investigate the intervention effects of five probiotics and their mix probiotics and metabolites thereof on colitis, we created a DSS-induced colitis model in 14-week-old C57BL/6J mice: 2.5% DSS was added to sterile water for free feeding for 7 days, followed by 7 days of free access to sterile water. At the same time, the mice in the 6 experimental groups were intragastically administered with corresponding single strain or mix strains daily which was suspended in filtered supernatant, while the control group was intragastically administered with PBS. Throughout the entire experiment, experiment indicators such as weight, changes in stool consistency, and blood in feces were recorded daily. In addition, fecal samples were collected on Day8 and Day 14 for gut microbiota analysis through 16S rRNA sequencing. Colon tissue was also collected for subsequent H&E. Staining and IHC experiments, etc., after sacrificing the mice (FIG. 9A). Through analysis, it was found that compared with the control group, the group of mix strains and metabolite thereof could improve the colonic shortening caused by DSS (FIG. 9B). In addition, compared with the control group, the weight change curve of the strain and metabolites thereof was flatter, and the weight loss was significantly reduced (FIG. 9C), and the DAI score was also significantly lower (FIG. 9D). The right bar charts in 9C and 9D, from left to right, are CONTROL, MIX, LG, LR1, LR2, LA, and BL. However, the single strain group showed less significant reduction in weight changes and DAI scores compared to the mix probiotic strains group. Overall, it can be seen that the mix strains group alleviated the pathological symptoms of DSS-induced experimental colitis.

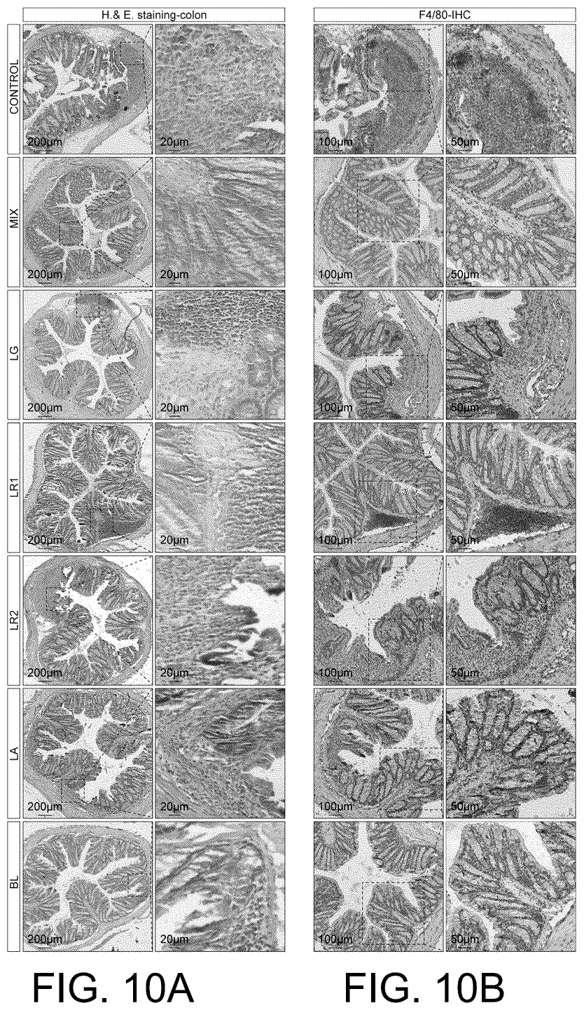

4. H.&E Staining was performed on colon sections to evaluate colonic mucosal damage. From the colon sections of the control group, changes in intestinal structure and barrier were observed, manifested as mucosal layer damage, crypt damage, and infiltration of inflammatory cells (FIG. 10A). In contrast, the treatment of mix strains and metabolites thereof protected the colon, manifested by intact crypts and less infiltration of inflammatory cells, as found by histological evaluation of colon sections. Macrophages have been identified as biomarkers for assessing the severity of colitis. In order to further investigate the effects of mix strains and metabolites thereof on colitis models, immunohistochemistry experiments (IHC) was used to detect macrophage markers (F4/80) associated with inflammation (FIG. 10B), and the treatment results were consistent with the result of H&E Staining, showing a significant decrease in the F4/80 levels of the strains and metabolites thereof, and the expression level of F4/80 in the single strain treatment group was also relatively low compared with the control group. In summary, the strains and metabolites thereof can restore inflammatory damage to the colon epithelium.

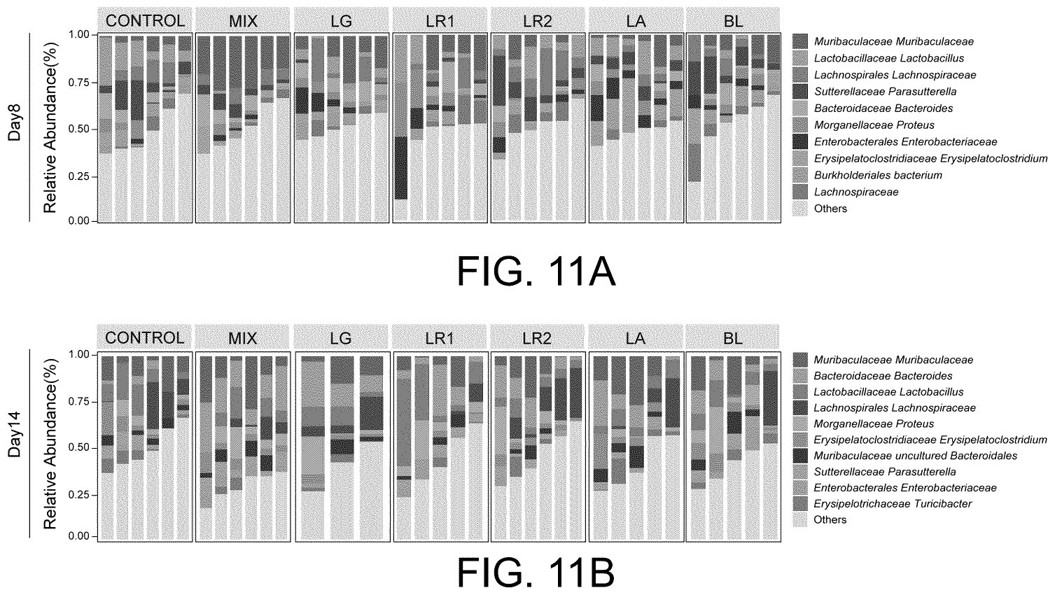

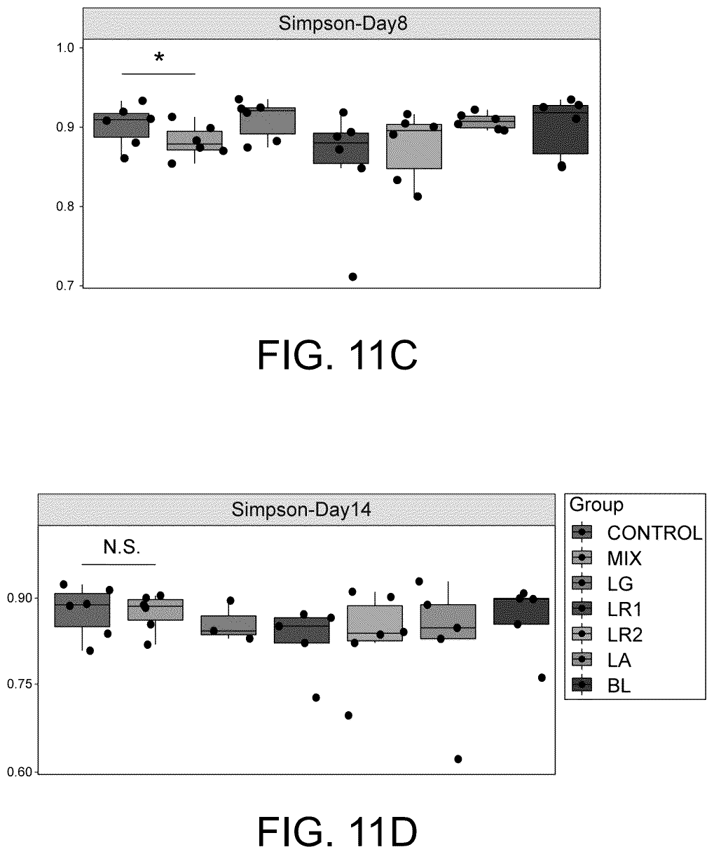

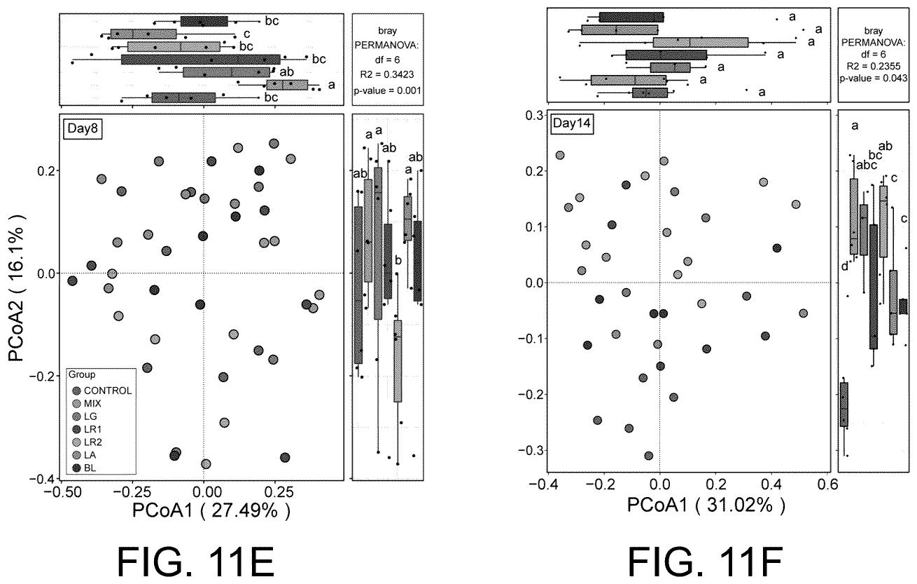

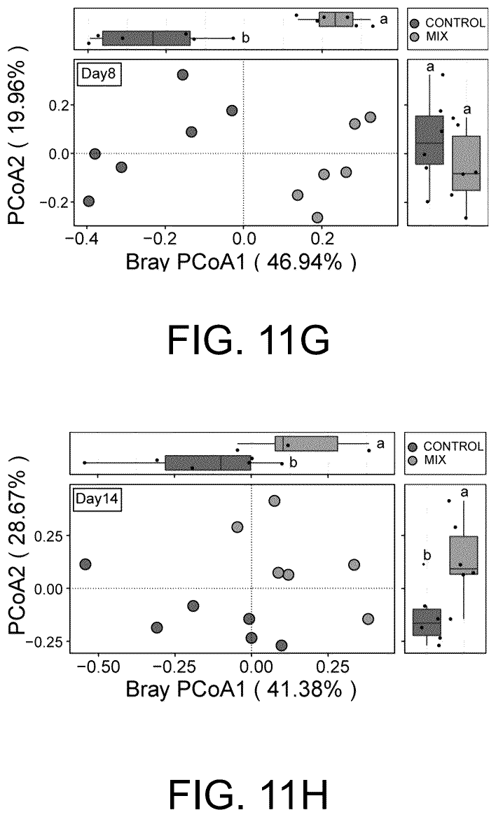

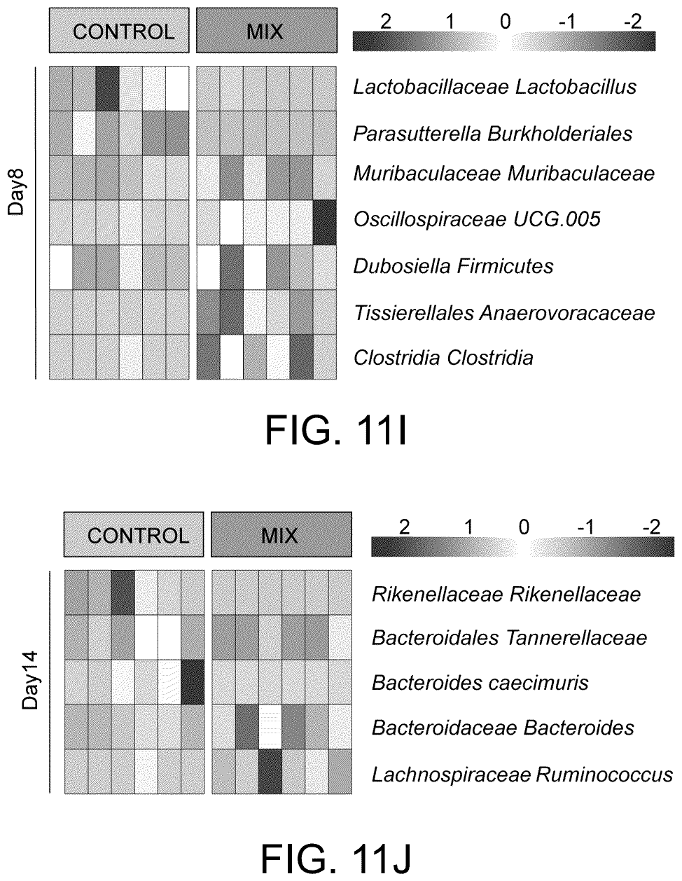

5. It was found that during different experiment periods, there were differences at the species level in the composition of the gut microbiota for the seven groups. Among the groups of strains and metabolites thereof, the relative abundance of Muribaculaceae Muribaculaceae was higher on Day 8, while that of Bacteroidaceae Bacteroides was higher on Day 14 (FIG. 11A, B). Meanwhile, the α-diversity values (Simpson index) of 7 groups at the species level were evaluated on Day 8 and Day 14. Among them, the Simpson index of the strains and metabolites thereof was significantly different from the control group on Day 8, while there was no significant difference in the other single strain treatment groups (FIG. 11C). There was no difference between the control group and the group of strains and metabolites thereof on Day14 (Simpson index, FIG. 11D). These results indicate that the intervention of mix strains and metabolites thereof can have a strong impact on the microbial community composition and diversity during inflammation, thereby further accelerating the recovery of inflammation. In addition, on Day8, the β-diversity based on Bray-Curtis metric distance principal coordinate analysis (PCoA) showed that only the gut microbiota of the control group and the group of mix strains and metabolites thereof were obviously divided into two colonies (FIG. 11E, G), indicating a significant change in the microbial structure of the mix strains and metabolites thereof at the species level on Day8. It is worth noting that on Day14, there were significant differences in PCoA graphs between the seven groups and the control group (FIG. 11F, H). In fact, in contrast, the mix strains and metabolites thereof showed different microbial compositions during the recovery of intestinal inflammation, for example, Muribaculaceae Muribaculaceae, Oscillospirales UCG.005 on Day8 and Bacteroidaceae Bacteroides, Lachnospiraceae Ruminococcus on Day14 (FIG. 11I-J). In summary, it can be seen that mix probiotic strains can regulate the gut microbiota composition of DSS-induced colitis models.

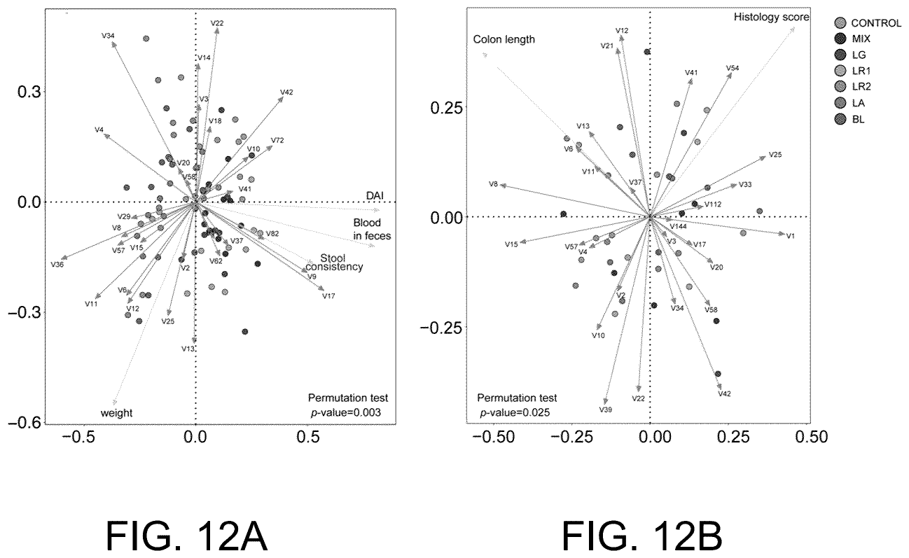

6. In our colitis and mix strains and metabolite thereof treatment models, disease phenotype data (such as weight, DAI, colon length, and histological score) and changes in gut microbiota (such as α-diversity, β-diversity and relative abundance) suggested that the host and gut microbiota may jointly contribute to the occurrence, progression, and recovery of the disease. Therefore, we utilized Redundancy Analysis (RDA) to unlock the relationship between gut microbiota composition and phenotype data. At the species level, there is a strong correlation between the seven groups of core microbiota and the phenotype data matrix (FIG. 12A: model p-value: 0.003, FIG. 12B: model p-value: 0.025). Among them, the determined variables composed of core microbial data at the taxonomic level jointly explained 52.68% of the variability in weight, DAI, blood in feces, and stool consistency among the seven sample groups. After 999 permutation tests, the constraints of bacteria V4, V9, V17, V36, V34, and V42 on the layout of this phenotype data were statistically significant (V4: r2-0.1152, p=0.016; V9: r2=0.1658, p=0.001; V17: r2=0.2226, p=0.001; V36: r2=0.2292, p=0.001; V34: r2=0.1557, p=0.003; V42: r2=0.1228, p=0.009). In addition, the determination of core microbial data at the taxonomic level for colon length and histological score was up to 89.13%, and V8, V39, V12, and V42 had a significant impact on this phenotype data (V8: r2=0.2082, p=0.028; V39: r2-0.1823, p=0.028; V12: r2-0.1673, p=0.045; V42: r2=0.1932, p=0.026). Overall, it can be seen that there is a strong correlation between gut microbiota and the disease phenotype of colitis mice models.

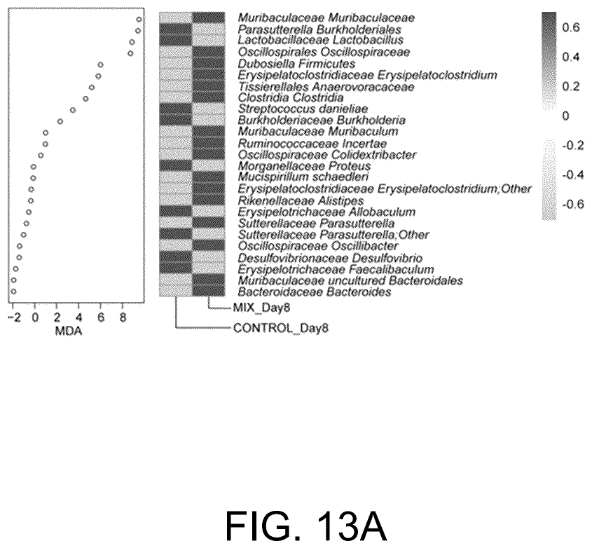

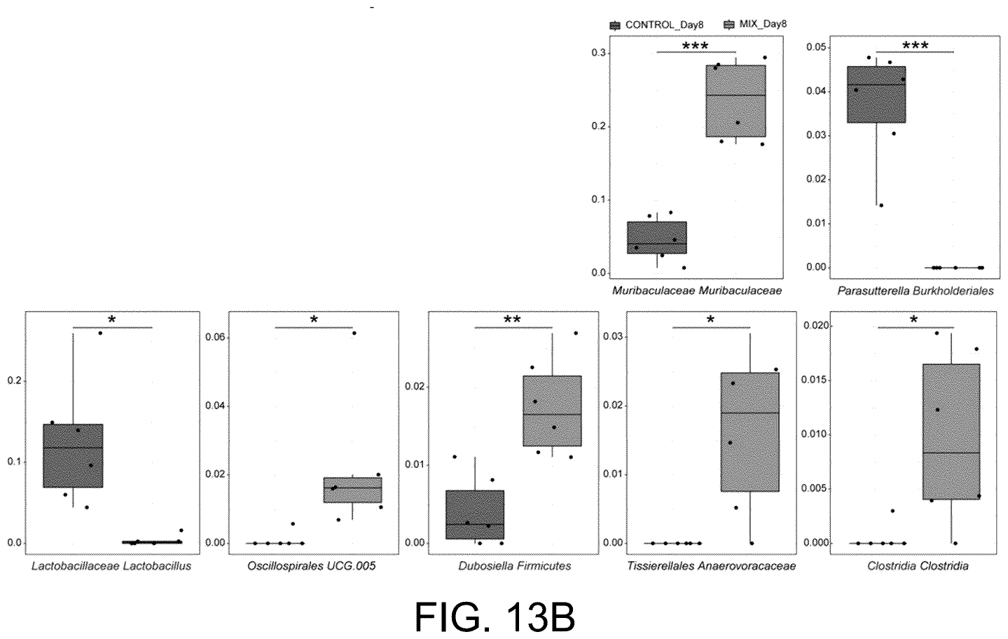

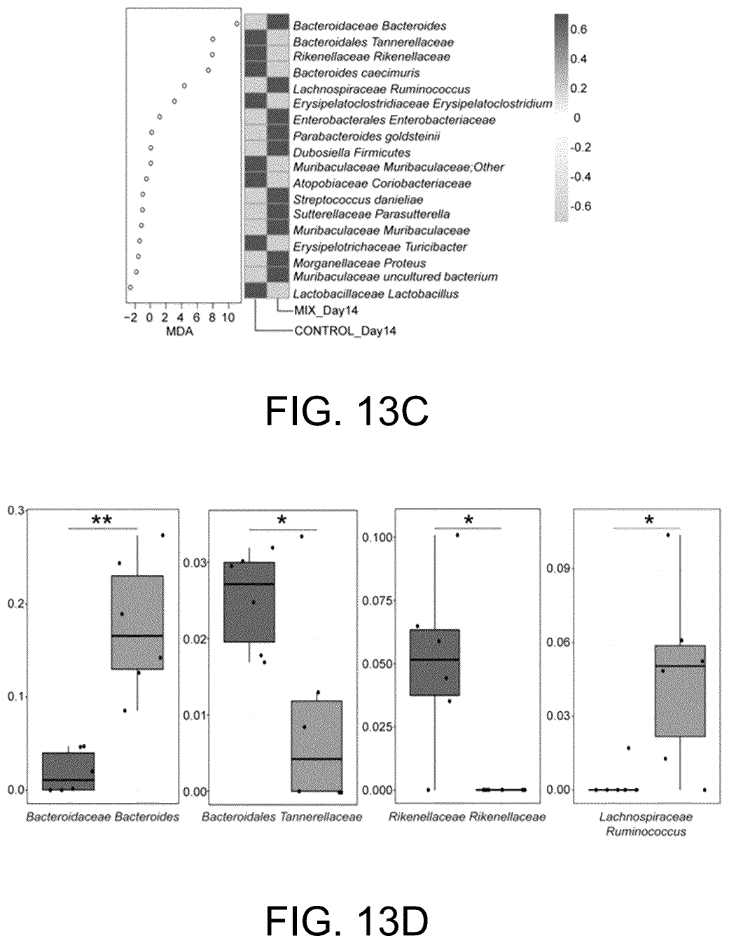

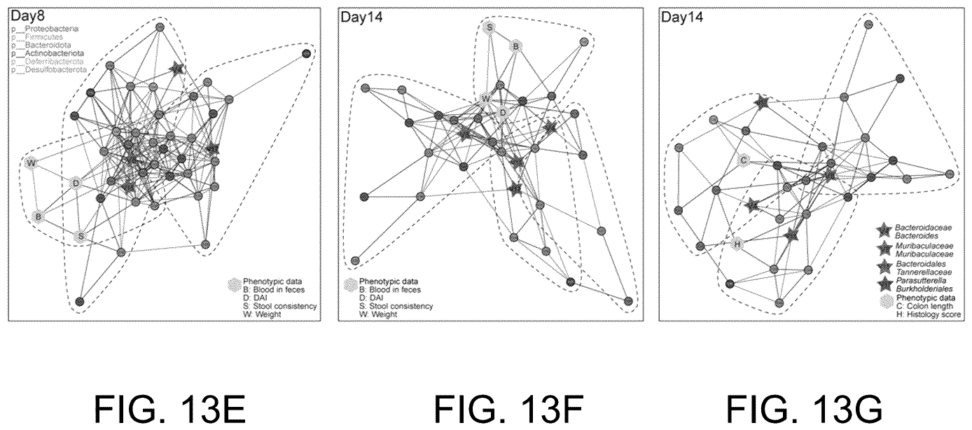

7. In order to accurately identify microbial markers during the recovery of intestinal inflammation, we mainly conducted random forest analysis combined with 5-fold cross validation on the group of mix strains and metabolites thereof and the control group on Day 8 and Day 14, respectively, to further identify characteristic bacteria (FIG. 13A, C). After analyzing the union of the two groups, it was found that eleven strains showed significant differences, with seven strains on Day 8 and four on Day 14. On Day 8, when comparing the two groups, the relative abundances of Parasottella Burkholderiales and Lactobacillusceae Lactobacillus in the control group were significantly increased, while the relative abundances of Muricaceae Muricaceae, Oscillospirales UCG.005, Dubosiella Firmicutes, Tissierellales Anaerovoracaceae, and Clostridia Clostridia in the mix group were significantly increased (FIG. 13B). In addition, on Day 14, the relative abundance of Bacteroidales Tannerellaceae and Rikenellaceae Rikenellacea significantly increased after the treatment of the control group, and the relative abundance of Bacteroidaceae Bacteroides and Lachnospiraceae Ruminococcus significantly increased after the treatment of mix strains and metabolites thereof (FIG. 13D). In the probiotic intervention group, the core strains and probiotic core strains Bacteroidales Tannerellaceae and Rikenellaceae Rikenellacea were located in the core region of the bacterial network on both Day 8 and Day 14 (FIG. 13E-G). The data on the intervention of probiotics indicated that the treatment of mix strains and metabolite thereof exerted a comprehensive effect through specific regulation of the intestinal microenvironment.

1. Use of one or more of Lactobacillus reuteri PLBK®1, Lactobacillus reuteri PLBK®2, Lactobacillus gasseri PLBK®3, Lactobacillus acidophilus PLBK®4 and Bifdobacterium lactis PLBK®5, fermentation cultures or metabolites thereof in the preparation of a product for alleviating colorectitis.

2. The use according to claim 1, wherein the product is food, healthcare products, or drugs.

3. The use according to claim 1, wherein the use is use of a mixture of five strains, the Lactobacillus reuteri PLBK®1, the Lactobacillus reuteri PLBK®2, the Lactobacillus gasseri PLBK®3, the Lactobacillus acidophilus PLBK*4 and the Bifdobacterium lactis PLBK®5, a mixture of the fermentation cultures or a mixture of the metabolites thereof in the preparation of the product for alleviating colorectitis.

4. The use according to claim 3, wherein the Lactobacillus reuteri PLBK®1, the Lactobacillus reuteri PLBK®2, the Lactobacillus gasseri PLBK®3, the Lactobacillus acidophilus PLBK®4 and the Bifdobacterium lactis PLBK®5 are mixed at a quantity ratio of 1:1:1:1:1.

5. The use according to claim 4, wherein the mixture of the Lactobacillus reuteri PLBK®1, the Lactobacillus reuteri PLBK®2, the Lactobacillus gasseri PLBK®3, the Lactobacillus acidophilus PLBK®4 and the Bifdobacterium lactis PLBK®5 is a bacterial solution and an amount of bacteria is 10^{\hat{ }9} cfu/ml and above.

6. The use according to claim 5, wherein the amount of the bacteria in the bacterial solution is 10^{\hat{ }9} cfu/ml.

7. Use of one or more of Lactobacillus reuteri PLBK®1, Lactobacillus reuteri PLBK®2, Lactobacillus gasseri PLBK®3, Lactobacillus acidophilus PLBK®4 and Bifdobacterium lactis PLBK®5, fermentation cultures or metabolites thereof in the preparation of a product for increasing relative abundance of Bacteroidaceae Bacteroides and Lachnospiraceae Ruminococcus.

8. Use of Bacteroidaceae Bacteroides and/or Lachnospiraceae Ruminococcus as biomarkers for identifying recovery of intestinal inflammation.

9. Use of a preparation for detecting an amount of Bacteroidaceae Bacteroides and/or Lachnospiraceae Ruminococcus in the preparation of a preparation for identifying recovery of intestinal inflammation.
